(19) United States
(12) Patent Application Publication
(10) Pub. No.: US 2016/0015654 A1
(43) Pub. Date: Jan. 21, 2016

(54) ADHESIVE COMPOSITION FOR MEDICAL USE, PATCH FOR MEDICAL USE, AND METHOD FOR PRODUCING THE COMPOSITION

(71) Applicant: TOAGOSEI CO., LTD., Tokyo (JP)

(72) Inventors: Shuhei YAMAGUCHI, Nagoya-shi (JP); Toshiyuki WAKAYAMA, Nagoya-shi (JP)

(73) Assignee: TOAGOSEI CO., LTD., Tokyo (JP)

(21) Appl. No.: 14/868,597

(22) Filed: Sep. 29, 2015

Related U.S. Application Data
(62) Division of application No. 13/641,042, filed on Dec. 17, 2012.

(30) Foreign Application Priority Data
Apr. 13, 2010 (JP) 2010-091926

(51) Int. Cl. A61K 9/70; A61K 9/00; C09J 133/08; A61K 31/192

(52) U.S. Cl. CPC A61K 9/7061; A61K 31/192; A61K 9/0014; C09J 133/08

(57) ABSTRACT

An adhesive composition for medical use of the present invention includes an acrylic copolymer, a medicament having a carboxyl group, and a base, the acrylic copolymer is prepared by copolymerization of a monomer mixture containing 0.1 to 2 mass % of an ethylenically unsaturated carboxylic acid monomer as a monomer A, 58 to 99.9 mass % of an alkyl(meth)acrylate ester having an alkyl group of 4 to 12 carbon atoms as a monomer B, 30 mass % or less of an alkyl(meth)acrylate ester having 1 to 3 carbon atoms as a monomer C, and 30 mass % or less of a monomer having an ethylenically unsaturated group other than those of the monomers A to C (the total of monomers A to D is taken as 100 mass %) as a monomer D, and the content of sulfuric acid in the acrylic copolymer is 700 ppm by mass or less.

### CROSS-REFERENCE TO RELATED APPLICATIONS

The present application is a 37 C.F.R. §1.53(b) divisional of U.S. application Ser. No. 13/641,042 filed Oct. 12, 2012, which is the 371 National Phase application of PCT International Application No. PCT/JP2011/058878 filed Apr. 8, 2011, which claims priority on Japanese Patent Application No. 2010-091926 filed Apr. 13, 2010. The entire contents of each of these applications is hereby incorporated by reference.

### TECHNICAL FIELD

The present invention relates to an adhesive composition for medical use that is used in applications for bonding on skin in the medical field, and a patch for medical use employing the adhesive composition.

### BACKGROUND ART

In the recent years, as a dermal administration means of medicaments, a patch for medical use such as a plaster medical preparation and poultice medical preparation have been in wide application. Such medical preparations are prepared by spreading an adhesive polymer containing a medicament on a substrate such as a nonwoven fabric, plastic film, etc. For the plaster medical preparation, an oil base prepared from a mixture of synthetic (or natural) rubber, various types of elastomers, oils, tackifiers, fragrances, etc. is used. For the poultice medical preparation, an aqueous base prepared by crosslinking a partially-neutralized acrylic acid copolymer by an aluminum compound, followed by mixing with glycerin, a pigment, and a fragrance, etc. is used.

An anti-inflammatory analgesic component such as ibuprofen, loxoprofen, ketoprofen, flurbiprofen, felbinac, diclofenac, etc. is added in a plaster medical preparation and a patch medical preparation. These medicaments have a carboxyl group in the molecule. However, as the carboxyl group make a dehydration condensing reaction with the other components or functional groups in the molecules of the medicament, a problem that the content of the medicament in the patch for medical use decreases during the shelf life is known. For example, Patent Document 1 describes that it is possible to suppress esterification of a carboxylic acid type non-steroid anti-inflammatory analgesic with 1-menthol by adding polyethylene glycol in a patch for medical use. Patent Document 2 describes that it is possible to suppress the intramolecular dehydration cyclization of diclofenac sodium by setting the relative humidity in a package of a patch for medical use within a predetermined range. Patent Document 3 describes that it is possible to suppress the esterification of ketoprofen with glycerin by not using glycerin as a feed material for preparing a patch for medical use.

On the other hand, an acrylic adhesive is widely used for a patch for medical use as an adhesive for plaster medical preparations and patch medical preparations. In the field of adhesives for medical use, their adhesions on skin, skin irritations, and solubility of the medicament, etc. have been mainly investigated. For example, Patent Document 4 describes that the amount of a residual organic peroxide-based initiator decreases and a change in the adhesive properties over time can be suppressed by polymerization at a high temperature for a long time to produce an adhesive.

### PRIOR ART DOCUMENTS

### Patent Documents

Patent Document 1: JP-A-2004-83462 (JP-A denotes a Japanese unexamined patent application publication)
Patent Document 2: JP-A-2008-61862
Patent Document 3: JP-A-2009-227640
Patent Document 4: JP-A-9-124467

### SUMMARY OF THE INVENTION

### Problems to be Solved by the Invention

However, the patch for medical use disclosed in the Patent Document 1 requires adding a new material. The patch for medical use disclosed in the Patent Document 2 requires strict humidity controls inside the package of the patch product. Consequently, these methods are not simple methods. In addition, the patch disclosed in the Patent Document 3 has insufficient moisture retention as it does not contain glycerin, and it is not a desired product from the viewpoint of low irritation on skin. As far as the adhesive for medical use disclosed in the Patent Document 4 is concerned, a stability of the medicament in the acrylic adhesive has not been investigated, and this is a problem for the adhesive composition in medical uses.

It is an object of the present invention to solve the aforementioned problems of the prior art by providing an adhesive composition for medical use which has excellent adhesion on the skin, and has little pain when peeled off, and in which adhesive composition, the medicament has high stability.

### Means for Solving the Problems

As a result of an intensive investigation by the present inventors in order to solve the aforementioned problems, it was found that in order to increase the stability of a medicament having a carboxyl group in its molecules, it is necessary to delay the progress in the esterification of the medicament contained in the adhesive composition. For this purpose, it has been found that it is effective to control the quantity of acidic substances as catalysts of dehydration reaction. Examples of the acidic substances contained in an acrylic adhesive include acrylic acid and methacrylic acid copolymerized for having adhesion, as well as a pH adjusting agent used in manufacturing the adhesive, decomposed substances of a polymerization initiator, etc.

That is, the present invention is as follows.

1. An adhesive composition for medical use containing an acrylic copolymer, a medicament having a carboxyl group, and a base, wherein the acrylic copolymer is prepared by copolymerization of a monomer mixture containing 0.1 to 2 mass % of an ethylenically unsaturated carboxylic acid monomer as a monomer A, 58 to 99.9 mass % of an alkyl(meth)acrylate ester having an alkyl group of 4 to 12 carbon atoms as a monomer B, 30 mass % or less of an alkyl(meth)acrylate ester having 1 to 3 carbon atoms as a monomer C, and 30 mass % or less of a monomer having an ethylenically unsaturated group other than those of the monomers A to C (the total of monomers A to D is taken as 100 mass %) as a monomer D, and the content of sulfuric acid in the acrylic copolymer is 700 ppm by mass or less.

2. The adhesive composition for medical use according to the aforementioned 1., wherein the acrylic copolymer is manufactured by emulsion polymerization, and the gel fraction thereof is 60% of higher.

3. The adhesive composition for medical use according to the aforementioned 1. or 2., wherein the medicament having a carboxyl group is an anti-inflammatory analogesic.

4. A adhesive sheet applying the adhesive composition for medical use according to any of the aforementioned 1. to 3. on one side of a substrate.

5. A method for producing an adhesive composition for medical use containing an acrylic copolymer, a medicament having a carboxyl group and a base, the method comprising a step of emulsion-polymerizing a monomer mixture containing an ethylenically unsaturated carboxylic acid monomer and an alkyl(meth)acrylate ester having an alkyl group of 4 to 12 carbon atoms by using a persulfate polymerization initiator to form the acrylic copolymer with a sulfuric acid content of 700 ppm or less.

### Effects of the Invention

The adhesive composition for medical use of the present invention has excellent adhesion on skin and causes little pain when peeled off, so it has excellent performances as an adhesive for medical use. Also, as there is little denaturing of the medicament in the adhesive composition, the stability of the medicinal effect is high.

In addition, when the acrylic copolymer related to the present invention is manufactured by emulsion polymerization and the gel fraction of the acrylic copolymer is 60% or higher, it can form an adhesive composition for medical use with excellent adhesion and cohesiveness.

In addition, as the adhesive composition for medical use of the present invention has a high stability of the medicinal effect, a shelf life of the patch for medical use can be prolonged.

### MODES FOR CARRYING OUT THE INVENTION

In the following, embodiments of the present invention will be explained in detail. The adhesive composition for medical use of the present invention is an adhesive composition for medical use containing an acrylic copolymer, a medicament having a carboxy group, and a base. The acrylic copolymer is prepared by copolymerization of specific monomers, and the content of sulfuric acid in the acrylic copolymer is 700 ppm by mass or less.

In the present specification, “(meth)acryl” refers to acryl or methacryl, and “(meth)acrylate” refers to acrylate or methacrylate.

### <Acrylic Copolymer>

The acrylic copolymer of the present invention is prepared by copolymerization of a monomer mixture containing 0.1 to 2 mass % of an ethylenically unsaturated carboxylic acid monomer as a monomer A, 58 to 99.9 mass % of an alkyl(meth)acrylate ester having an alkyl group of 4 to 12 carbon atoms as a monomer B, 30 mass % or less of an alkyl(meth)acrylate ester having 1 to 3 carbon atoms as a monomer C, and 30 mass % or less of a monomer having an ethylenically unsaturated group other than those of the monomers A to C (the total of monomers A to D is taken as 100 mass %) as a monomer D.

Examples of the ethylenically unsaturated carboxylic acid monomer (monomer A) include unsaturated monobasic acids such as acrylic acid, methacrylic acid, crotonic acid, vinyl acetic acid, acryloxy propionic acid and the like, unsaturated dibasic acids such as maleic acid, itaconic acid, fumaric acid, mesaconic acid, citraconic acid, cyclohexane dicarboxylic acid and the like, unsaturated acid anhydrides such as maleic anhydride, itaconic anhydride, citraconic anhydride, tetrahydrophthalic anhydride and the like, etc. Among them, the acrylic acid and methacrylic acid are preferable as these compounds can easily make copolymerization reactions with various other types of monomers and are inexpensive. The monomer A may be used singly or in a combination of two or more compounds.

The content of the ethylenically unsaturated carboxylic acid monomer (monomer A) in the monomer mixture is in the range of 0.1 to 2.0 mass %, preferably in the range of 0.2 to 2.0 mass %, and more preferably in the range of 0.5 to 1.8 mass % (the total of monomers A to D is 100 mass %). If the content is less than 0.1 mass %, the adhesive strength of the obtained adhesive composition is low, separation takes place easily during the bonding period, and residual paste may be left when it is peeled off. On the other hand, if the content is over 2.0 mass %, progress of esterification of the medicament in the adhesive composition goes fast, the quantity of the effective medicament decreases, so the shelf life of the patch for medical use becomes shorter.

Examples of the alkyl(meth)acrylate ester (monomer B) having an alkyl group of 4 to 12 carbon atoms include n-butyl(meth)acrylate, isobutyl(meth)acrylate, sec-butyl(meth)acrylate, tert-butyl(meth)acrylate, n-pentyl(meth)acrylate, isoamyl(meth)acrylate, n-hexyl(meth)acrylate, 2-methylpentyl(meth)acrylate, n-octyl(meth)acrylate, iso-octyl(meth)acrylate, 2-ethylhexyl(meth)acrylate, n-nonyl(meth)acrylate, isononyl(meth)acrylate, 2-methyloctyl(meth)acrylate, decyl(meth)acrylate, dodecyl(meth)acrylate, tridecyl(meth)acrylate, lauryl(meth)acrylate, etc. Among them, the n-butyl(meth)acrylate and 2-ethylhexyl(meth)acrylate are preferable, as these compounds can easily perform copolymerization reaction with various other types of monomers, and are inexpensive. Monomer B may be used singly or in a combination of two or more compounds.

The content of the alkyl(meth)acrylate ester having an alkyl group of 4 to 12 carbon atoms (monomer B) is in the range of 58 to 99.9 mass %, preferably in the range of 65 to 99.8 mass %, and more preferably in the range of 75 to 99.5 mass % (the total of the monomers A to D is 100 mass %). If the content is less than 58 mass %, the adhesive strength of the obtained adhesive composition is low, separation takes place easily during the bonding period, and residual paste may be left when it is peeled off. On the other hand, if the content is over 99.9 mass %, the adhesive strength of the obtained adhesive composition is too high and skin irritation becomes strong.

Examples of the alkyl(meth)acrylate ester having 1 to 3 carbon atoms (monomer C) include methyl(meth)acrylate, ethyl(meth)acrylate, n-propyl(meth)acrylate, i-propyl(meth)acrylate, etc. Among them, methyl(meth)acrylate and ethyl(meth)acrylate are preferable. Monomer C may be used singly or in a combination of two or more compounds.

The content of the alkyl(meth)acrylate ester having 1 to 3 carbon atoms (monomer C) is 30 mass % or less, preferably in the range of 5 to 25 mass %, and more preferably in the range of 10 to 20 mass % (the total of the monomers A to D is 100 mass %). If the content is over 30 mass %, the adhesive strength of the obtained adhesive composition is low, separation takes place easily during the bonding period, and residual paste may be left when it is peeled off.

Examples of the monomer having an ethylenically unsaturated group other than those of the monomers A to C (monomer D) include a vinyl monomer having a cyano group, a vinyl monomer having a hydroxyl group, an aromatic vinyl monomer, an alicyclic vinyl monomer, a vinyl monomer having an amino group, a vinyl monomer having an amido group, a vinyl monomer having an alkoxyl group, a vinyl monomer having a carboxyl group, a conjugated diene monomer, a maleimide monomer, a vinyl ester monomer, a vinyl ether monomer, a vinyl monomer having a glycidyl group, a mono- or di-alkyl ester of unsaturated dicarboxylic acid, an unsaturated alcohol, a chlorine-containing vinyl monomer, a multi-vinyl monomer, a monomer having a silicon-containing group, etc. Monomer D may be used singly or in a combination of two or more compounds.

Examples of the vinyl monomer having a cyano group include acrylonitrile, methacrylonitrile, α-ethyl acrylonitrile, α-isopropyl acrylonitrile, α-chloro acrylonitrile, α-fluoro acrylonitrile, etc. These compounds may be used singly or in a combination of two or more types.

Examples of the vinyl monomer having a hydroxyl group include 2-hydroxyethyl(meth)acrylate, 2-hydroxypropyl(meth)acrylate, 3-hydroxypropyl(meth)acrylate, 2-hydroxybutyl(meth)acrylate, 3-hydroxybutyl(meth)acrylate, 4-hydroxybutyl(meth)acrylate, 6-hydroxyhexyl(meth)acrylate, 8-hydroxyoctyl(meth)acrylate, 12-hydroxylauryl(meth)acrylate, 2-hydroxy-3-phenoxypropyl acrylate, 2-hydroxy-3-p-methylphenoxypropyl acrylate, 2-hydroxy-3-2-ethylhexyloxypropyl acrylate, mono(meth)acrylate esters of polyalkylene glycol such as polyethylene glycol, polypropylene glycol, and p-hydroxy styrene, m-hydroxy styrene, o-hydroxy styrene, p-isopropenyl phenol, m-isopropenyl phenol, o-isopropenyl phenol, etc. These compounds may be used singly or in a combination of two or more types.

Examples of the aromatic vinyl monomer include styrene, 2-methyl styrene, 3-methyl styrene, 4-methyl styrene, α-methyl styrene, 2,4-dimethyl styrene, 2,4-diisopropyl styrene, 4-tert-butyl styrene, tert-butoxy styrene, vinyl toluene, divinyl toluene, benzyl(meth)acrylate, vinyl naphthalene, monochloro styrene, dichloro styrene, mono-bromo styrene, di-bromo styrene, tri-bromo styrene, fluoro styrene, styrene sulfonic acid and salts thereof, α-methyl styrene sulfonic acid and salts thereof, etc. These compounds may be used singly or in a combination of two or more types.

Examples of the alicyclic vinyl monomer include cyclohexyl(meth)acrylate, methylcyclohexyl(meth)acrylate, t-butylcyclohexyl(meth)acrylate, cyclododecyl(meth)acrylate, isobornyl(meth)acrylate, etc. These compounds may be used singly or in a combination of two or more types.

Examples of the vinyl monomer having an amino group include amino ethyl(meth)acrylate, dimethylaminomethyl(meth)acrylate, diethylaminomethyl(meth)acrylate, 2-dimethylaminoethyl(meth)acrylate, 2-diethylaminoethyl(meth)acrylate, 2-(di-n-propylamino)ethyl(meth)acrylate, 2-dimethylaminopropyl(meth)acrylate, 2-diethylaminopropyl(meth)acrylate, 2-(di-n-propylamino)propyl(meth)acrylate, 3-dimethylaminopropyl(meth)acrylate, 3-diethylaminopropyl(meth)acrylate, 3-(di-n-propylamino)propyl(meth)acrylate, etc. These compounds may be used singly or in a combination of two or more types.

Examples of the vinyl monomer having an amido group include (meth)acrylamide, N-methyl(meth)acrylamide, N,N-dimethyl(meth)acrylamide, N,N-dimethylaminopropyl(meth)acrylamide, N,N-butoxymethyl(meth)acrylamide, N-methylol(meth)acrylamide, N-alkoxymethyl(meth)acrylamide, etc. These compounds may be used singly or in a combination of two or more types.

Examples of the vinyl monomer having an alkoxy group include 2-methoxyethyl(meth)acrylate, 2-ethoxyethyl(meth)acrylate, 2-(n-propoxy)ethyl(meth)acrylate, 2-(n-butoxy)ethyl(meth)acrylate, 3-methoxypropyl(meth)acrylate, 3-ethoxypropyl(meth)acrylate, 2-(n-propoxy)propyl(meth)acrylate, 2-(n-butoxy)propyl(meth)acrylate, etc. These compounds may be used singly or in a combination of two or more types.

Examples of the vinyl monomer having a carbonyl group include (meth)acrolein, diacetone(meth)acrylamide, formistyrol, (meth)acryloxy alkylpropanal, diacetone(meth)acrylate, acetonyl(meth)acrylate, acetoacetoxyethyl(meth)acrylate, acetoacetoxy allyl ester, 2-hydroxypropyl(meth)acrylate-acetyl acetate, butanediol-1,4-acrylate-acetyl acrylate, vinyl methyl ketone, vinyl ethyl ketone, vinyl isobutyl ketone, etc. These compounds may be used singly or in a combination of two or more types.

Examples of the conjugated diene monomer include 1,3-butadiene, isoprene (2-methyl-1,3-butadiene), 2,3-dimethyl-1,3-butadiene, chloroprene (2-chloro-1,3-butadiene), etc. These compounds may be used singly or in a combination of two or more types.

Examples of the maleimide monomer include maleimide, N-methyl maleimide, N-isopropyl maleimide, N-butyl maleimide, N-dodecyl maleimide, N-phenyl maleimide, N-(2-methylphenyl) maleimide, N-(4-methylphenyl)maleimide, N-(2,6-dimethylphenyl) maleimide, N-(2,6-diethylphenyl) maleimide, N-(2-methoxyphenyl) maleimide, N-benzyl maleimide, N-(4-hydroxyphenyl) maleimide, N-naphthyl maleimide, N-cyclohexyl maleimide, etc. These compounds may be used singly or in a combination of two or more types.

Examples of the vinyl ester monomer include methylene aliphatic monocarboxylic acid ester, vinyl acetate, vinyl propionate, vinyl pivalate, vinyl butyrate, vinyl benzoate, vinyl formate, vinyl cinnamate, vinyl versatate, etc. These compounds may be used singly or in a combination of two or more types.

Examples of the vinyl ether monomer include vinyl methyl ether, vinyl ethyl ether, vinyl-n-butyl ether, vinyl isobutyl ether, vinyl phenyl ether, vinyl cyclohexyl ether, etc. These compounds may be used singly or in a combination of two or more types.

Examples of the vinyl monomer having a glycidyl group include glycidyl(meth)acrylate, (meth)allyl glycidyl ether, β-methyl glycidyl(meth)acrylate, 4-hydroxybutyl glycidyl(meth)acrylate, 3,4-epoxycyclohexylmethyl(meth)acrylate, 3,4-epoxycyclohexylethyl(meth)acrylate, 3,4-epoxycyclohexylpropyl(meth)acrylate, etc. These compounds may be used singly or in a combination of two or more types.

Examples of the monoalkyl ester of unsaturated dicarboxylic acid include monoalkyl esters of maleic acid, fumaric acid, itaconic acid, citraconic acid, mesaconic acid, maleic anhydride, itaconic anhydride, citraconic anhydride, or tetrahydrophthalic anhydride, etc. These compounds may be used singly or in a combination of two or more types.

Examples of the dialkyl ester of unsaturated dicarboxylic acid include dialkyl esters of maleic acid, fumaric acid, itaconic acid, citraconic acid, mesaconic acid, maleic anhydride, itaconic anhydride, citraconic anhydride, tetrahydrophthalic acid, etc. These compounds may be used singly or in a combination of two or more types.

Examples of unsaturated alcohol include allyl alcohol, methallyl alcohol, 3-methyl-3-buten-1-ol, 3-methyl-2-buten-1-ol, 2-methyl-3-buten-2-ol, etc. These compounds may be used singly or in a combination of two or more types.

Examples of the chlorine-containing vinyl monomer include vinyl chloride, vinylidene chloride, etc. These compounds may be used singly or in a combination of two or more types.

Examples of the multi-vinyl monomer include allyl(meth)acrylate, ethylene glycol di(meth)acrylate, triethylene glycol di(meth)acrylate, tetraethylene glycol di(meth)acrylate, 1,3-butylene glycol di(meth)acrylate, trimethylol propane tri(meth)acrylate, 1,4-butanediol di(meth)acrylate, neopentyl glycol di(meth)acrylate, 1,6-hexanediol di(meth)acrylate, pentaerythritol di(meth)acrylate, pentaerythritol tetra(meth)acrylate, glycerol di(meth)acrylate, 1,1,1-trishydroxymethylethane di(meth)acrylate, 1,1,1-trishydroxymethylethane tri(meth)acrylate, 1,1,1-trishydroxymethylpropane tri(meth)acrylate, triallyl isocyanurate, diallyl phthalate, divinyl benzene, etc. These compounds may be used singly or in a combination of two or more types.

Examples of the monomer having a silicon-containing group include vinyl trichlorosilane, vinyl tribromosilane, vinyl trimethoxy silane, vinyl triethoxy silane, vinyl tri-n-propoxy silane, vinyl tri-i-propoxy silane, vinyl tri-n-butoxy silane, vinyl tris(2-hydroxymethoxyethoxy) silane, vinyl triacetoxy silane, vinyl diethoxy silanol, vinyl ethoxy silanediol, vinyl methyl diethoxy silane, vinyl dimethyl ethoxy silane, vinyl methyl diacetoxy silane, allyl trimethoxy silane, allyl triethoxy silane, 3-methacryloxypropyl trimethoxy silane, 3-acryloxypropyl triethoxy silane, 3-methacryloxypropyl tris(2-methoxyethoxy) silane, 3-methacryloxypropyl methyl diethoxy si lane, 3-methacryloxypropyl dimethyl ethoxy silane, 3-acryloxy propyl dimethoxy silane, 2-acrylamideethyl triethoxy silane, etc. These compounds may be used singly or in a combination of two or more types.

Among the monomers having an ethylenically unsaturated group (monomer D), a vinyl monomer having a cyano group, an aromatic vinyl monomer and a vinyl ester monomer are preferable. By using these monomers, it is possible to manufacture the acrylic copolymer with excellent adhesion and cohesiveness at a low price.

The content of the monomer having an ethylenically unsaturated group (monomer D) is 30 mass % or less, preferably 15 mass % or less, and more preferably 5 mass % or less.

### <Production Method of the Acrylic Copolymer>

The acrylic copolymer can be manufactured using any of known methods, such as emulsion polymerization, solution polymerization, suspension polymerization, etc. Among them, the emulsion polymerization and solution polymerization are preferable as the polymerization time is short and the manufacturing operation is simple.

### Emulsion Polymerization

There is no specific restriction on the method for emulsion polymerization. For example, any of the following listed methods may be used: (1) a method in which a monomer and a water-based medium are loaded in a polymerization device beforehand, and, after setting at the predetermined temperature, a polymerization initiator is added, (2) a method in which a water-based medium is loaded in a polymerization device beforehand, and, after setting at the predetermined temperature, a monomer and a polymerization initiator are added, and (3) a method in which a water-based medium and a portion of a monomer are loaded in a polymerization device beforehand, and, after setting at the predetermined temperature, the remaining portion of a monomer and a polymerization initiator are added.

The amount of the water-based medium for emulsion polymerization with respect to the total of 100 parts by mass of the monomers is preferably in the range of 10 to 1000 parts by mass, and more preferably in the range of 50 to 200 parts by mass. Examples of the water-based medium include water alone, a mixture of water and alcohol, etc. Among them, water is preferable as it is not a hazardous material.

The polymerization initiator used in emulsion polymerization may be a radical polymerization initiator such as a peroxide, an azo compound, etc. A redox polymerization initiator of a reducing agent, such as ascorbic acid, sodium ascorbate, sodium erythorbate, tartaric acid, citric acid, metal salts of formaldehyde sulfoxylate, sodium thiosulfate, sodium sulfite, sodium bisulfite, sodium metabisulfite, ferric chloride, etc., together with peroxide can be used.

Examples of peroxides include inorganic peroxides such as hydrogen peroxide, persulfate salts such as sodium persulfate, ammonium persulfate, and potassium persulfate, etc. An organic peroxides may be used. Examples of organic peroxides include hydroperoxides such as cumene hydroperoxide, para-menthanehydroperoxide, tert-butyl hydroperoxide, etc.; dialkyl peroxides such as tert-butyl cumyl peroxide, dicumyl peroxide, etc.; diacyl peroxides; peroxy esters such as tert-butyl peroxy laurate, tert-butyl peroxy benzoate, etc.; benzoyl peroxide, lauroyl peroxide, peracetic acid, and persuccinic acid. These peroxides may be used singly or in a combination of two or more types.

Examples of the azo compound include 2,2′-azobisisobutyronitrile, 2,2′-azobis(2,4-dimethylvaleronitrile), 2,2′-azobis(2-methylbutyronitrile), 2,2′-azobis(4-methoxy-2,4-dimethylvaleronitrile), 2,2′-azobis(2-amidinopropane)dihydrochloride, 2,2′-azobis[N-(2-carboxyethyl)-2-methylpropione diamine]tetrahydrate salt, etc. These azo compounds may be used singly or in a combination of two or more types.

The content of the polymerization initiator in emulsion polymerization is selected appropriately corresponding to the type of the polymerization initiator and the polymerization conditions, etc., usually, and is preferably 0.001 to 10 parts by mass with respect to 100 parts by mass of the monomer mixture.

An emulsifier may be adopted in emulsion polymerization.

Examples of the known emulsifiers that can be used in the conventional emulsion polymerization include various types of emulsifiers such as anionic emulsifiers, nonionic emulsifiers, cationic emulsifiers, amphoteric emulsifiers, etc. Examples of the anionic emulsifiers include dialkyl sulfosuccinate salt, alkyl benzenesulfonate salt, alkyl sulfate salt, polyoxyethylene alkyl phenyl ether sulfate salt, polyoxyethylene alkyl diphenyl ether sulfate salt, polyoxyethylene alkyl ether sulfate salt, alkyl diphenyl ether disulfonate salt, polymeric emulsifier, etc. Examples of the nonionic emulsifiers include polyoxyethylene higher alcohol ether, polyoxyethylene alkyl phenyl ether, polyoxyethylene alkyl diphenyl ether, polyoxyethylene-polyoxypropylene block copolymer, acetylene diol emulsifier, sorbitan higher fatty acid esters, polyoxyethylene sorbitan higher fatty acid esters, polyoxyethylene higher fatty acid esters, glycerin higher fatty acid ester, polycarboxylic acid polymeric emulsifier, polyvinyl alcohol, etc. Examples of the cationic emulsifiers include alkyl(amido)betaine, alkyl dimethylamine oxide, and special emulsifiers such as fluoro emulsifiers and silicone emulsifiers, etc. These emulsifiers may be used singly or in a combination of two or more types.

In addition to the non-reactive emulsifiers mentioned above, reactive emulsifiers may also be used. The reactive emulsifier refers to an emulsifier having polymerizable functional groups such as ethylenically unsaturated groups, etc. There is no specific restriction on the type of the reactive emulsifier for use in this case. Any emulsifier having reactive groups may be used. Examples of the reactive emulsifier include the emulsifiers represented by general formulae (1) to (12) below. These may be used singly or in a combination of two or more types.

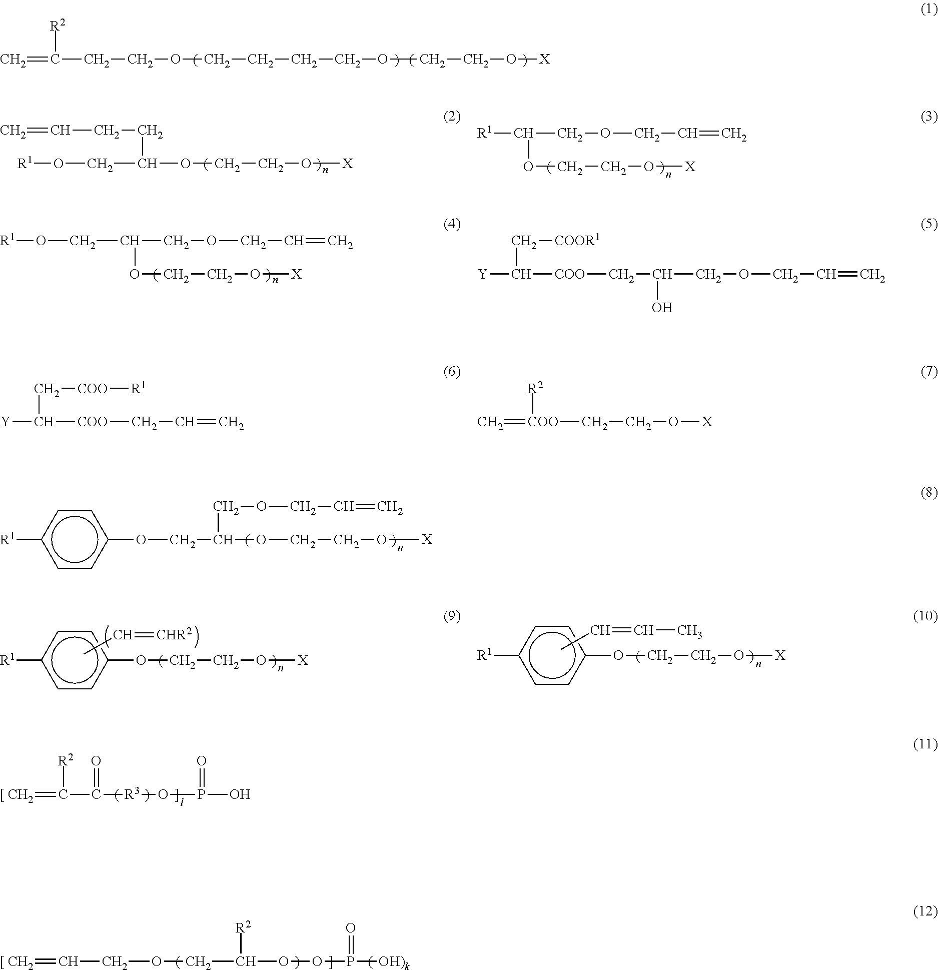

In the general formulae (1) to (12), R¹ represents an alkyl group, R² represents a hydrogen atom or methyl group, R³ represents an alkylene group, n and m represent integers of 1 or larger, 1 and k represent integers of 1 or larger (l+k=3), X represents a hydrogen atom, SO₃NH₄ or SO₃Na, and Y represent SO₃NH₄ or SO₃Na.

The amount of the emulsifier used is selected appropriately corresponding to the type of the emulsifier and the polymerization condition, etc., and usually, with respect to 100 parts by mass of the monomer mixture, is preferably in the range of 0.1 to 50 parts by mass, more preferably in the range of 0.3 to 30 parts by mass, and particularly preferably in the range of 0.5 to 20 parts by mass.

In the emulsion polymerization, a chain transfer agent (molecular weight adjusting agent) or the like may be used.

Examples of the chain transfer agent include mercapto group-containing compounds (ethanethiol, butanethiol, dodecanethiol, benzenethiol, toluenethiol, α-toluenethiol, phenethyl mercaptan, mercaptoethanol, 3-mercaptopropanol, thioglycerin, thioglycolic acid, 2-mercaptopropionic acid, 3-mercaptopropionic acid, α-mercaptoisobutyric acid, methyl mercaptopropionate, ethyl mercaptopropionate, thioacetic acid, thiomalic acid, thiosalicylic acid, octyl mercaptan, n-dodecyl mercaptan, tert-dodecyl mercaptan, n-hexadecyl mercaptan, n-tetradecyl mercaptan, tert-tetradecyl mercaptan, etc.), xanthogen disulfide compounds (dimethyl xanthogen disulfide, diethyl xanthogen disulfide, diisopropyl xanthogen disulfide, etc.), thiuram disulfide compounds (tetramethyl thiuram disulfide, tetraethyl thiuram disulfide, tetrabutyl thiuram disulfide, etc.), halogenated hydrocarbons (carbon tetrachloride, ethylene bromide, etc.), aromatic hydrocarbons (pentaphenyl ethane, α-methyl styrene dimer, etc.), etc. These compounds may be used singly or in a combination of two or more types.

The amount of the chain transfer agent is selected appropriately corresponding to the type of the chain transfer agent and the polymerization condition, and usually, with respect to 100 parts by mass of the monomer mixture, is preferably in the range of 0.001 to 1 part by mass.

The polymerization temperature of the emulsion polymerization is selected appropriately according to the types of monomers and the type of the radical polymerization initiator, etc., usually, and is preferably in the range of 10 to 100° C., more preferably in the range of 30 to 98° C., and particularly preferably in the range of 50 to 95° C.

For the acrylic copolymer obtained in the emulsion polymerization, the pH may be adjusted by adding an acidic substance or a basic substance.

Examples of the acidic substance include hydrochloric acid, sulfuric acid, nitric acid, methane sulfonic acid, benzene sulfonic acid, p-toluene sulfonic acid, phosphoric acid, acetic acid, tartaric acid, malic acid, citric acid, benzoic acid, etc. These compounds may be used singly or in a combination of two or more types. These compounds may be added directly into the acrylic copolymer, or may be dissolved in water and then added as a water solution.

Examples of the basic substances include alkali metal compounds (sodium hydroxide, potassium hydroxide, etc.), alkaline earth metal compounds (calcium hydroxide, calcium carbonate, etc.), ammonia, organic amine compounds (monomethylamine, dimethylamine, trimethylamine, monoethylamine, diethylamine, methylethylamine, triethylamine, monopropylamine, dimethylpropylamine, monoethanolamine, diethanolamine, triethanolamine, ethylene diamine, diethylene triamine, etc.), etc. These compounds may be used singly or in a combination of two or more types. These compounds may be added as is in the acrylic copolymer, or may be dissolved in water and then added as a water solution.

When the acrylic copolymer is prepared by emulsion polymerization, the gel fraction is preferably 60% or more, or more preferably 65% or more. When the gel fraction is set in this way, it is possible to obtain an acrylic copolymer with excellent adhesion and cohesiveness.

The gel fraction can be measured using a high speed solvent extracting device. For example, a predetermined amount of the dry film prepared from the acrylic copolymer is weighed precisely to prepare a test sample, and Ottawa sand is mixed with the test sample prepared from the dry film to obtain a sample. The obtained sample is set in the high speed solvent extracting device, and the sample is subjected to extraction by ethyl acetate. The liquid extracted from the sample is dried and ethyl acetate is removed to obtain the ethyl acetate extract.

The gel fraction can be calculated using the following formula from the mass of the obtained ethyl acetate extract and the mass of the test substance.

Gel fraction (mass %)=((mass of the test sample (g)−mass of the ethyl acetate extract (g))/mass of the test sample (g))×100

### Solution Polymerization

The solution polymerization is usually carried out as follows. In a polymerization vessel, the predetermined organic solvent, monomers and polymerization initiator, and optionally an chain transfer agent are loaded together. Then, in a nitrogen gas flow or at the reflux temperature of the organic solvent, reaction is carried out while heated with stirring for a few hours. In this case, at least a portion of the organic solvent, monomers, polymerization initiator and/or chain transfer agent may be added in sequence.

Examples of the organic solvents that may be adopted in the solution polymerization include aliphatic or alicyclic hydrocarbons such as benzene, toluene, ethylbenzene, n-propylbenzene, t-butylbenzene, o-xylene, m-xylene, p-xylene, tetralin, decalin, aromatic naphtha, and other aromatic hydrocarbons; n-hexane, n-heptane, n-octane, i-octane, n-decane, dipentene, petroleum spirit, petroleum naphtha, turpentine oil, etc.; ketones such as ethyl acetate, n-butyl acetate, n-amyl acetate, 2-hydroxyethyl acetate, 2-butoxyethyl acetate, 3-methoxybutyl acetate, methyl benzoate, and other esters; acetone, methyl ethyl ketone, methyl i-butyl ketone, isophorone, cyclohexanone, methyl cyclohexanone, etc.; glycol ethers such as ethylene glycol monomethyl ether, ethylene glycol monoethyl ether, ethylene glycol monobutyl ether, diethylene glycol monomethyl ether, diethylene glycol monoethyl ether, diethylene glycol monobutyl ether, etc.; alcohols such as methyl alcohol, ethyl alcohol, n-propyl alcohol, i-propyl alcohol, n-butyl alcohol, i-butyl alcohol, s-butyl alcohol, t-butyl alcohol, etc.; etc. These organic solvents may be used singly or in a combination of two or more types.

A polymerization initiator used in solution polymerization may be used a compound that the polymerization initiator used in emulsion polymerization above-mentioned. A chain transfer agent used in solution polymerization may be used a compound that the chain transfer agent used in emulsion polymerization above-mentioned, too.

The polymerization temperature in solution polymerization is selected appropriately corresponding to the types of the monomers and the type of the radical polymerization initiator, usually, and is preferably in the range of 10 to 180° C., more preferably in the range of 30 to 150° C., and yet more preferably in the range of 50 to 120° C.

When unreacted monomers are contained in the acrylic copolymer obtained in the solution polymerization, in order to remove the unreacted monomers, it is possible to carry out purification by means of a re-precipitation method using methanol or the like.

When the acrylic copolymer is prepared by means of solution polymerization, the gel fraction of the acrylic copolymer is preferably 20% or lower, and more preferably 15% or lower. If the gel fraction is set in this way, it is possible to obtain an acrylic copolymer with excellent adhesion and cohesiveness.

The content of the sulfuric acid in the acrylic copolymer prepared using emulsion polymerization, solution polymerization or other polymerization method, is 700 ppm by mass or less, and preferably 300 ppm by mass or less. The sulfuric acid in the acrylic copolymer comes from the polymerization initiator and pH adjusting agent adopted in manufacturing the acrylic copolymer. If the content of sulfuric acid in the acrylic copolymer is over 700 ppm, the esterification reaction of the medicament makes progress quickly, the quantity of effective medicament decreases, so the shelf life of the patch for medical use becomes shorter.

The content of sulfuric acid in the acrylic copolymer described in the present specification is determined by measuring the concentration of sulfuric acid ion by ion chromatography to be explained later. The value is then converted to the value of the sulfuric acid.

### <Medicament Having Carboxyl Group>

The adhesive composition for medical use of the present invention contains a medicament having a carboxyl group. There is no specific restriction on the type of medicament of the present invention, as long as it is a compound having a carboxyl group and a pharmaceutically tolerable salt of the compound, such as anti-inflammatory analgesics, antibiotics, etc. Examples of the pharmaceutically tolerable salts of the medicament include alkali metal salts, alkaline earth metal salts, ammonium compounds, etc., and specific examples include sodium, potassium, calcium, magnesium, ammonia, dimethylamine, diethylamine, trimethylamine, tetramethyl ammonium, monoethanolamine, diethanolamine, triethanolamine, etc. The specific examples of the medicament are listed below.

(a) Anti-inflammatory analgesics: Salicylic acid, sulindac, naproxen, fenbufen, indomethacin, ketoprofen, mefenamic acid, flufenamic acid, ibufenac, loxoprofen, thiaprofen, pranoprofen, diclofenac, alclofenac, ibuprofen, felbinac, bermoprofen, naproxen, flurbiprofen, etc.

(b) Antibiotics: penicillin, benzyl penicillin, methicillin, oxacillin, cloxacillin, ampicillin, amoxicillin, bacampicillin, talampicillin, ticarcillin, azocillin, mezlocillin, piperacillin, carbenicillin, etc.

### <Base>

The adhesive composition for medical use of the present invention contains a base. The base ensures adhesion on the skin, shape retention when applied, and the continuous supply of the medicament to the skin. The bases include a non-aqueous base and an aqueous base to be explained below.

### Non-Aqueous Base

Examples of the components that form the non-aqueous base include a rubber polymer, a tackifier, a plasticizer, etc.

Examples of the rubber polymer include styrene-isoprene copolymers, styrene-butadiene copolymers, polyisobutylene, polyisoprene, polybutene, silicone rubber copolymers, crude rubber, etc. These rubber polymers may be used singly or in a combination of two or more types.

Examples of the tackifier include coumarone-indene resin, terpene resin, terpene-phenolic resin, rosin resin, p-t-butyl phenol-acetylene resin, phenol-formaldehyde resin, xylene-formaldehyde resin, petroleum hydrocarbon resin, hydrogenated hydrocarbon resin, turpentine resin, etc. These tackifiers may be used singly or in a combination of two or more types.

Examples of the plasticizer include petroleum oils (paraffin process oil, naphthene process oil, aromatic process oil, etc.), squalane, squalene, plant-base oils (olive oil, camellia oil, castor oil, tall oil, peanut oil), silicone oil, dibasic acid esters (dibutyl phthalate, dioctyl phthalate, etc.), liquid rubber (polybutene, liquid isoprene rubber), liquid fatty acid esters (isopropyl myristate, hexyl laurate, diethyl sebacate, diisopropyl sebacate), diethylene glycol, polyethylene glycol, glycol salicylate, propylene glycol, dipropylene glycol, triacetene, triethyl citrate, crotamiton, etc. These plasticizers may be used singly or in a combination of two or more types.

### Aqueous Base

Examples of the ingredients that form the aqueous base include a water soluble polymer, a crosslinking agent, a polyhydric alcohol, water, etc.

Examples of the water soluble polymer include polyacrylic acid, polyacrylate salt, partially neutralized substance of polyacrylic acid, polyacrylamide, polyethylene imine, polyvinyl alcohol, polyvinyl pyrrolidone, carboxyvinyl polymer, methyl cellulose, carboxymethyl cellulose, sodium carboxymethyl cellulose, hydroxyethyl cellulose, starch acrylate, vinyl ethylacetate, gelatin, starch, eudragit, alginic acid, sodium alginate, tragacanth, etc. These water soluble polymer may be used singly or in a combination of two or more types.

As the crosslinking agent, salts that generate bivalent or trivalent metal ions when dissolved in water may be utilized. Examples include hydroxides such as aluminum hydroxide, aluminum magnesium hydroxide, and the like, salts of inorganic acids and organic acids, and basic salts thereof such as aluminum chloride, aluminum sulfate, dihydroxy aluminum amino acetate, kaolin, aluminum stearate, magnesium hydroxide, magnesium chloride, magnesium sulfate, and the like, double salts such as aluminum alum and the like, aluminate salts such as sodium aluminate and the like, inorganic aluminum complex salts, organic aluminum chelate compounds, synthetic hydrotalcite, magnesium alminometasilicate, magnesium alminosilicate, aluminum nitrate, aluminum sulfate, EDTA-aluminum, aluminum allantoinate, aluminum acetate, aluminum glycinal, etc. These crosslinking agents may be used singly or in a combination of two or more types.

The salts that generate bivalent or trivalent metal ions used as crosslinking agents may be either water soluble salts or water sparingly soluble salts. When an water sparingly soluble aluminum compound is used as the crosslinking agent, a reaction rate adjusting agent may be added in the reaction system where gelling should be carried out. Especially, by adding an acid, it is possible to increase the reaction rate of gelling. In particular, by adding an organic acid containing hydroxyl groups or the salts of the acid, it is possible to significantly increase the gelling reaction rate. Examples of the reaction rate adjusting agents that may be adopted in this case include organic acids, organic acid salts, organic bases that have a chelate forming ability or coordination ability with respect to the metal ions such as citric acid, lactic acid, tartaric acid, gluconic acid, glycolic acid, malic acid, fumaric acid, metasulfonic acid, maleic acid, acetic acid, EDTA-disodium, urea, triethylamine, ammonia, etc., and inorganic acids such as hydrochloric acid, phosphoric acid, sulfuric acid, nitric acid, hydrobromic acid, etc.

Examples of the polyhydric alcohols include ethylene glycol, propylene glycol, trimethylene glycol, 1,3-butanediol, ethylene glycol monobutyl ether, triethylene glycol, 1,4-butanediol, glycerin, trioxy isobutane, erythrit, pentaerythrit, xylit, adonit, allitol, sorbitol, sorbitol liquid, mannitol, polyethylene glycol, etc.

As explained above, the adhesive composition for medical use of the present invention contains an acrylic copolymer, a medicament having a carboxyl group and a base. The contents of the components are as follows. When it is used in a plaster medical preparation, with respect to the total mass of the ingredients, the content of the acrylic copolymer is preferably in the range of 1 to 50 mass %, the content of the medicament having a carboxyl group is preferably in the range of 0.01 to 10 mass %, and the content of the non-aqueous base is preferably in the range of 50 to 99 mass %. The content of the acrylic copolymer is more preferably in the range of 3 to 30 mass %, the content of the medicament having a carboxyl group is more preferably in the range of 0.1 to 5 mass %, and the content of the non-aqueous base is more preferably in the range of 70 to 96 mass %. On the other hand, when it is used for a poultice medical preparation, with respect to the total mass, the content of the acrylic copolymer is preferably in the range of 1 to 20 mass %, the content of the medicament having a carboxyl group is preferably in the range of 0.01 to 10 mass %, and the content of the aqueous base is preferably in the range of 80 to 99 mass %. It is preferred that the content of the acrylic copolymer is more preferably in the range of 3 to 15 mass %, the content of the medicament having a carboxyl group is more preferably in the range of 0.1 to 5 mass %, and the content of the aqueous base is more preferably in the range of 85 to 97 mass %.

The adhesive composition for medical use of the present invention may also contain other additives as long as the purpose of the present invention can be realized. Examples of the other additives include a dermal absorption accelerating agent, a tackifier, a softening agent, an oxidation inhibitor, an anti-aging agent, a preservative, a fragrance, a pH adjustor, an emulsifier, a disperser, a stabilizer, an excipient, a dissolving agent, etc.

### <Patch for Medical Use>

The adhesive composition for medical use of the present invention may be directly applied on a substrate to form a patch for medical use. A scheme can be adopted in which once the adhesive composition for medical use is applied on a release paper, it is transferred to the substrate.

Examples of substrates that may be adopted in this case include polyethylene, polypropylene, ethylene/vinyl acetate copolymer, polystyrene, polyester, polyvinyl chloride, polyvinylidene chloride, polyurethane, polyamide, and various types of plastic films. Also woven fabric, knitware, nonwoven fabric, paper, metal foil, and laminates thereof may be used.

The thickness of the substrate depends on the type of the substrate, usually, the thickness is preferably 500 μm or thinner, and more preferably in the range of 40 to 200 μm. There is no specific restriction on the thickness of the adhesive composition for medical use, and the thickness is usually in the range of 20 to 1000 μm.

### EXAMPLES

In the following, the present invention will explained in more detail with reference to examples. However, the present invention is not limited to these examples. In the following explanation, “parts” and “%” refer to those by mass unless otherwise specified.

### 1. Manufacturing of the Acrylic Copolymer

### <Manufacturing Example 1>

29 parts of water were loaded in a reaction vessel equipped with a stirrer, a reflux condenser, a dropping tank, a nitrogen introduction tube, and a thermometer, and the temperature was kept at 70° C. In the dropping tank, 25 parts of water, 2 parts of polyoxyethylene nonyl phenyl ether (EO addition 30 mol), 1 part of acrylic acid, 14 parts of methyl acrylate, and 85 parts of 2-ethylhexyl acrylate were loaded, and are stirred to form an emulsion.

While the emulsion was continuously added dropwise into the reaction vessel over 7 h, 0.1 parts of 1% aqueous solution of ammonium persulfate as polymerization initiator was added dropwise over 7.5 h for emulsion polymerization. Then, after aging for 1 h, t-butyl hydroperoxide and sodium erythorbate were added to reduce the unreacted monomers to obtain an acrylic copolymer. The solid content, viscosity, pH, gel fraction, and sulfuric acid concentration of the obtained acrylic copolymer were analyzed under the following conditions.

a. Solid Content

1 g of the sample was measured into an aluminum cup. After drying in a drier at 155° C. for 30 min, the weight was measured, and the solid content was calculated.

b. Viscosity

The viscosity was measured using a BM type viscometer at 12 rpm and 25° C.

c. pH

The pH was measured using a pH meter at 25° C.

d. Gel Fraction

(i) Preparation of a Sample

In a box made of a release paper (15 cm in length x 7.5 cm in width x 3 cm in height), the acrylic copolymer of an amount corresponding to a thickness of the resulting dried film of 1 mm was poured. Then, the acrylic copolymer was dried at room temperature (about 18° C.) for 1 week to form a dry film.

(ii) Extraction by Ethyl Acetate

Extraction for the obtained dry film with ethyl acetate was carried out using a high speed solvent extracting apparatus ASE-200 (produced by of Nippon Dionex K.K.). More specifically, about 0.3 g of the obtained dry film was weighed precisely to form a test substance. The test sample was mixed with Ottawa sand, and the mixture was filled in an 11 mL extracting cell. Then, the extracting cell filled with the test sample and Ottawa sand was set in the high speed solvent extracting apparatus, and extraction was carried out twice repeatedly under the following condition. Then, the liquid extracted from the extracting cell was dried at 100° C. for 16 h to remove the ethyl acetate, and an ethyl acetate extract was obtained.

### Extracting Conditions

PREHEAT: 0 min, HEAT: 6 min, STATIC: 10 min, FLUSH %: 100 vol, PURGE: 90 sec, CYCLES: 3, PRESSURE: 1500 psi, TEMPERATURE: 120° C.

The gel fraction was calculated using the following formula.

Gel fraction (mass %)=[mass of the test sample (g)−mass of the ethyl acetate extract (g)]/mass of test sample (g)×100

e. Content of Sulfuric Acid in the Solid Content of the Acrylic Copolymer

After 0.5 g of the obtained acrylic copolymer was diluted 10 times by ultra-pure water with electrical resistivity of 18.2 M□·m or higher, ultrafiltration was carried out using USY-1 manufactured by AdvanTech K.K. The concentration of sulfate ions in the filtrate was analyzed by ion chromatography. From the analysis value of the concentration of sulfate ions in the filtrate, the content of sulfuric acid contained in the solid content of the acrylic copolymer was calculated (detection limit at 10 wtppm).

Apparatus: Dionex ICS Model 1000

Column: AS9-HC manufactured by Nippon Dionex K.K.

Column temperature: 35° C.

Detector: Electroconductivity detector

Mobile phase: Sodium carbonate of 0.009 mol/L

### <Manufacturing Examples 2 to 14>

In each manufacturing example, an acrylic copolymer was prepared in the same procedure as in Manufacturing Example 1, except that the types and quantities of the monomers and the polymerization initiator in use, and the pH adjustment method for the obtained copolymer were changed. The results are listed in Table 1. The abbreviations of the polymerization initiators adopted and listed in Table 1 are defined as follows.

APS: Ammonium persulfate

KPS: Potassium persulfate

AIBN: Azobisisobutyronitrile

### <Manufacturing Example 15>

50 parts of ethyl acetate and 30 parts of toluene were loaded in a reaction vessel equipped with a stirrer, a reflux condenser, a dropping tank, a nitrogen introduction tube, and a thermometer, and it was heated to about 75° C. inside the reactor vessel with nitrogen gas flow. Then, a monomer solution as a mixture of 1 part of acrylic acid, 14 parts of methyl acrylate, and 85 parts of 2-ethyl hexyl acrylate, and a solution of polymerization initiator prepared beforehand from a mixture of 1.0 part of azobisisobutyronitrile and 10 parts of ethyl acetate was added dropwise into the system over 3 h. Then, the system was kept at the same temperature for 5 h, and the polymerization reaction came to an end. Then, ethyl acetate was added to adjust the solid content to about 40% and the acrylic copolymer was obtained. Measurement of the solid content, viscosity, pH and gel fraction of the obtained acrylic copolymer was carried out in the same procedure as in Manufacturing Example 1. The content of sulfuric acid was measured using the following method.

### (Content of Sulfuric Acid in the Solid Content of the Acrylic Copolymer)

After 1.0 g of the obtained acrylic copolymer was diluted by 20 mL of ethyl acetate, an extraction operation was carried out by 10 mL ultra-pure water with electrical resistivity of 18.2 M□·m or higher. The concentration of sulfate ions in the water phase was analyzed by ion chromatography. From the analysis value of the concentration of sulfate ions in the water phase, the content of sulfuric acid contained in the solid content of the acrylic copolymer was calculated.

### <Comparative Manufacturing Examples 1 to 7>

In each example, a copolymer was prepared in the same procedure as in Manufacturing Example 1, except that the types and quantities of the monomers and polymerization initiator used and the pH adjustment method of the obtained copolymer were changed. The results are listed in Table 2.

### 2. Manufacturing of Adhesive Composition for Medical Use (for Poultice Medical Preparation)

### Example 1

A mixture liquid comprising 2 g of loxoprofen sodium dihydrate, 270.5 g of water, and 1 g of tartaric acid, and a dispersion comprising 40 g of sodium polyacrylate polymer and 150 g of glycerin were blended. Then, 35 g of the acrylic copolymer obtained by Manufacturing Example 1 and 1.5 g of aluminum hydroxide gel were added, and the mixture was homogeneously blended to obtain an adhesive composition for medical use.

### Examples 2 to 14

In these examples, adhesive compositions for medical use were prepared in the same procedure as in Example 1, except that the acrylic copolymers prepared in Manufacturing Examples 2 to 14 were used, instead of the acrylic copolymer prepared in Manufacturing Example 1.

### 3. Manufacturing of Poultice for Medical Preparation

After each adhesive composition obtained by Examples 1 to 14 was applied and spread in a coating amount of 1000 g/m² on a polyester nonwoven fabric, a polyethylene terephthalate film was bonded on the coated surface to obtain a poultice medical preparation. The obtained poultice medical preparation was cut to a predetermined size and subjected to the skin adhesion test and medicament stability test described below.

### 4. Manufacturing of Adhesive Composition for Medical Use (for Plaster Medical Preparation)

### Example 15

36 g of fluidic paraffin, 35 g of the acrylic copolymer obtained by Manufacturing Example 15, 40 g of alicyclic saturated hydrocarbon resin, 3 g of butyl hydroxy toluene, 5 g of loxoprofen and 250 g of toluene were added in 31 g of styrene-isoprene-styrene block copolymer, and the mixture was stirred and homogeneously blended to form a plaster solution as an adhesive composition for medical use (for plaster medical preparation).

### 5. Manufacturing of Plaster Medical Preparation

The adhesive composition for medical use obtained by Example 15 was applied on a release paper in an amount of the adhesive composition for medical use corresponding to 30 g/m² after drying. After drying, it was transferred to a polyester film to form a patch for medical use. The obtained patch was cut to the predetermined size for the following listed skin adhesion test and the medicament stability test.

### Examples 16 to 18, 20 to 22

Adhesive compositions for medical use were prepared in the same procedure as in Example 1, except that the type of the acrylic copolymer and the type of the medicament used were changed. As a result, adhesive compositions for medical use (for patch medical preparation) were obtained.

### Examples 19, 23

Adhesive compositions for medical use were prepared in the same procedure as in Example 15, except that the type of the medicament used was changed. As a result, adhesive compositions for medical use (for plaster medical preparation) were obtained.

### Comparative Examples 1 to 9

Adhesive compositions for medical use (for poultice medical preparation) were prepared in the same procedure as in Example 1, except that the type of the acrylic copolymer and the type of the medicament adopted were changed.

The types of the acrylic copolymers and the types of the medicaments used in Examples 1 to 23 and Comparative Examples 1 to 9 are listed in the columns of “acrylic copolymer” and “medicament” in Tables 3 through 6. In Tables 3 to 6, in the “medicament” column, “a” stands for loxoprofen sodium dihydrate, “b” stands for ketoprofen, and “c” stands for ibuprofen.

### 6. Test Method of Adhesive Composition

(1) Skin Adhesion

The patch medical preparation for medical use as the sample (poultice medical preparation or plaster medical preparation) is applied on human skin for 24 h. The state during application and the feeling in peeling are evaluated according to the following standards. In Tables 3 to 6, the column of “skin adhesion” and the column of “pain in peeling” show the skin adhesion of the patch medical preparation and the pain in peeling, respectively.

(a) Skin Adhesion
Excellent: Reliably bonded
Good: A little weak adhesion, yet no problem
Poor: Weak adhesion

(b): Pain when Peeling
Excellent: No pain
Good: A little pain, yet no problem
Poor: Pain is felt

(2) Medicament Stability Test

The aforementioned patch for medical use is stored at 60° C. for 1 month. After that, the content of the medicament in each patch for medical use is measured by means of high speed liquid chromatography (HPLC). In the column of “residual rate of medicament” in Tables 3 to 6, the residual rate (%) after a shelf time of 1 month with respect to the initial value of the content of the medicament contained in each patch for medical use, is listed.

### Condition for HPLC

Apparatus: Shimadzu LC-10A

Column: Toso ODS-80TM

Column temperature: 40° C.

Detecting wavelength: UV 225 nm

Mobile phase: Acetonitrile/water/phosphoric acid=40:60:0.02

TABLE 1

| | | Manufacturing Examples | | | | | | | |
|---|---|---|---|---|---|---|---|---|---|
| | | 1 | 2 | 3 | 4 | 5 | 6 | 7 | 8 |
| Monomer | (A) Acrylic acid | 1 | 0.2 | 1 | 0.5 | 1.8 | 1 | 2 | |
| (parts) | Methacrylic acid | | | | | | | | 1 |
| | (B) n-butyl acrylate | | | | | | | | |
| | 2-ethylhexyl acrylate | 85 | 85.8 | 85 | 85.5 | 84.2 | 74 | 93 | 85 |
| | (C) Methyl acrylate | 14 | 14 | 14 | 14 | 14 | 25 | 5 | 14 |
| | Ethyl acrylate | | | | | | | | |
| | (D) Vinyl acetate | | | | | | | | |
| | Acetonitrile | | | | | | | | |
| | Styrene | | | | | | | | |
| | Adjustment of pH with 10 wt % | — | — | — | — | — | — | — | — |
| | sulfuric acid | | | | | | | | |
| | Type of polymerization initiator | APS | APS | KPS | APS | APS | APS | APS | APS |
| | Quantity of polymerization initiator | 0.1 | 0.02 | 0.1 | 0.1 | 0.1 | 0.1 | 0.3 | 0.1 |
| | (parts) | | | | | | | | |
| Obtained acrylic copolymer | | (1) | (2) | (3) | (4) | (5) | (6) | (7) | (8) |
| Properties | Concentration of solid content (%) | 59.8 | 59.8 | 59.3 | 59.8 | 59.0 | 57.8 | 59.6 | 58.1 |
| and | Viscosity (mPa · s) | 144 | 140 | 100 | 405 | 300 | 120 | 125 | 420 |
| evaluation | pH | 2.5 | 2.9 | 2.4 | 2.7 | 2.4 | 2.7 | 2.2 | 2.6 |
| | Content of sulfuric acid (wtppm) | 380 | 50 | 290 | 230 | 321 | 340 | 652 | 357 |
| | Gel fraction (%) | 72 | 85 | 70 | 82 | 74 | 76 | 66 | 81 |

| | | Manufacturing Examples | | | | | | |
|---|---|---|---|---|---|---|---|---|
| | | 9 | 10 | 11 | 12 | 13 | 14 | 15 |
| Monomer | (A) Acrylic acid | 1 | | 1 | 1 | 1 | 1 | 1 |
| (parts) | Methacrylic acid | | 2 | | | | | |
| | (B) n-butyl acrylate | | 98 | 74 | | | 89 | |
| | 2-ethylhexyl acrylate | 85 | | | 79 | 90 | | 85 |
| | (C) Methyl acrylate | 14 | | | | 5 | | 14 |
| | Ethyl acrylate | | | 25 | | | | |
| | (D) Vinyl acetate | | | | 20 | | | |
| | Acetonitrile | | | | | 4 | | |
| | Styrene | | | | | | 10 | |
| | Adjustment of pH with 10 wt % | done | — | — | — | — | — | — |
| | sulfuric acid | | | | | | | |
| | Type of polymerization initiator | APS | APS | APS | APS | APS | APS | AIBN |
| | Quantity of polymerization initiator | 0.1 | 0.1 | 0.1 | 0.1 | 0.1 | 0.1 | 1 |
| | (parts) | | | | | | | |
| Obtained acrylic copolymer | | (9) | (10) | (11) | (12) | (13) | (14) | (15) |
| Properties | Concentration of solid content (%) | 57.3 | 57.3 | 58.1 | 56.0 | 54.0 | 59.0 | 40.2 |
| and | Viscosity (mPa · s) | 64 | 112 | 250 | 86 | 55 | 531 | 1150 |
| evaluation | pH | 1.7 | 3.1 | 2.9 | 2.8 | 2.8 | 2.7 | — |
| | Content of sulfuric acid (wtppm) | 682 | 156 | 298 | 250 | 356 | 176 | ND |
| | Gel fraction (%) | 75 | 76 | 71 | 83 | 90 | 65 | 0 |

TABLE 2

| | | Comparative | | | | | | |
|---|---|---|---|---|---|---|---|---|
| | | Manufacturing Examples | | | | | | |
| | | 1 | 2 | 3 | 4 | 5 | 6 | 7 |
| Monomer | (A) Acrylic acid | 3 | 1 | 3 | 5 | 0 | 1 | 1 |
| (parts) | Methacrylic acid | | | | | | | |
| | (B) n-butyl acrylate | | | | | | 50 | 50 |
| | 2-ethylhexyl acrylate | 83 | 85 | 83 | 81 | 86 | | |
| | (C) Methyl acrylate | 14 | 14 | 14 | 14 | 14 | 49 | |
| | Ethyl acrylate | | | | | | | |
| | (D) Vinyl acetate | | | | | | | 49 |
| | Acetonitrile | | | | | | | |
| | Styrene | | | | | | | |
| | Adjustment of pH with 10 wt % sulfuric | — | — | — | done | — | done | — |
| | acid | | | | | | | |
| | Type of polymerization initiator | APS | APS | APS | APS | APS | APS | APS |
| | Quantity of polymerization initiator (parts) | 0.1 | 0.5 | 0.5 | 1 | 0.1 | 0.1 | 0.1 |
| Obtained acrylic copolymer | | (16) | (17) | (18) | (19) | (20) | (21) | (22) |
| Properties | Concentration of solid content (%) | 59.7 | 60.1 | 59.8 | 55.6 | 59.5 | 59.7 | 56.1 |
| and | Viscosity (mPa · s) | 183 | 280 | 175 | 130 | 380 | 420 | 255 |
| evaluation | pH | 2.6 | 2.1 | 2 | 1.6 | 2.8 | 1.7 | 2.4 |
| | Content of sulfuric acid (wtppm) | 450 | 1230 | 810 | 3115 | 180 | 2650 | 360 |
| | Gel fraction (%) | 72 | 85 | 54 | 43 | 75 | 81 | 80 |

TABLE 3

| | Examples | | | | | | | |
|---|---|---|---|---|---|---|---|---|
| | 1 | 2 | 3 | 4 | 5 | 6 | 7 | 8 |
| Acrylic | (1) | (2) | (3) | (4) | (5) | (6) | (7) | (8) |
| copolymer | | | | | | | | |
| Medicament | a | a | a | a | a | a | a | a |
| Skin | Excellent | Good | Excellent | Excellent | Excellent | Excellent | Excellent | Excellent |
| adhesion | | | | | | | | |
| Pain when | Excellent | Excellent | Excellent | Excellent | Excellent | Excellent | Good | Excellent |
| peeled | | | | | | | | |
| Medicament | 97.1 | 99.2 | 97.4 | 98.0 | 95.4 | 96.9 | 94.8 | 97.1 |
| residual rate | | | | | | | | |
| (%) | | | | | | | | |

TABLE 4

| | Examples | | | | | | |
|---|---|---|---|---|---|---|---|
| | 9 | 10 | 11 | 12 | 13 | 14 | 15 |
| Acrylic | (9) | (10) | (11) | (12) | (13) | (14) | (15) |
| copolymer | | | | | | | |
| Medicament | a | a | a | a | a | a | a |
| Skin adhesion | Excellent | Excellent | Good | Good | Excellent | Excellent | Excellent |
| Pain when | Excellent | Good | Excellent | Excellent | Excellent | Excellent | Excellent |
| peeled | | | | | | | |
| Medicament | 95.9 | 96.8 | 96.8 | 97.1 | 96.8 | 97.5 | 99.4 |
| residual rate | | | | | | | |
| (%) | | | | | | | |

TABLE 5

| | Examples | | | | | | | |
|---|---|---|---|---|---|---|---|---|
| | 16 | 17 | 18 | 19 | 20 | 21 | 22 | 23 |
| Acrylic | (3) | (10) | (11) | (15) | (3) | (10) | (11) | (15) |
| copolymer | | | | | | | | |
| Medicament | b | b | b | b | c | c | c | c |
| Skin | Excellent | Excellent | Good | Excellent | Excellent | Excellent | Good | Excellent |
| adhesion | | | | | | | | |
| Pain when | Excellent | Good | Excellent | Excellent | Excellent | Good | Excellent | Excellent |
| peeled | | | | | | | | |
| Medicament | 96.9 | 95.2 | 96.5 | 99.5 | 95.4 | 94.3 | 94.3 | 98.1 |
| residual rate | | | | | | | | |
| (%) | | | | | | | | |

TABLE 6

| | Comparative Examples | | | | | | | | |
|---|---|---|---|---|---|---|---|---|---|
| | 1 | 2 | 3 | 4 | 5 | 6 | 7 | 8 | 9 |
| Acrylic | (16) | (17) | (18) | (19) | (20) | (21) | (22) | (19) | (19) |
| coploymer | | | | | | | | | |
| Medicament | a | a | a | a | a | a | a | b | c |
| Skin | Excellent | Excellent | Excellent | Excellent | Poor | Poor | Poor | Excellent | Excellent |
| adhesion | | | | | | | | | |
| Pain when | Poor | Excellent | Poor | Poor | Excellent | Excellent | Excellent | Poor | Poor |
| peeled | | | | | | | | | |
| Medicament | 92.8 | 90.8 | 90.4 | 89.7 | 97.0 | 93.7 | 97.4 | 90.5 | 87.3 |
| residual rate | | | | | | | | | |
| (%) | | | | | | | | | |

### INDUSTRIAL APPLICABILITY

As explained above, for the adhesive composition for medical use of the present invention, as it contains a prescribed acrylic copolymer, a medicament having a carboxyl group, and a base, it has excellent skin adhesion, and has little pain when peeled off. In addition, the medicament stability is high. Consequently, the patch for medical use using the adhesive composition for medical use of the present invention can be efficiently used in treating sprains, stiffness in shoulders, lumbago, joint pain, nerve pain, etc.

What is claimed is:

1. A method for producing an adhesive composition for medical use containing an acrylic copolymer, a medicament having a carboxyl group and a base, the method comprising:
a step of preparing a monomer mixture containing an ethylenically unsaturated carboxylic acid monomer and an alkyl(meth)acrylate ester having an alkyl group of 4 to 12 carbon atoms, and
a step of emulsion polymerizing the monomer mixture by using a persulfate polymerization initiator to form the acrylic copolymer with a sulfuric acid content of 700 ppm or less.

2. The method for producing an adhesive composition for medical use according to claim 1, wherein the monomer mixture containing 0.1 to 2 mass % of an ethylenically unsaturated carboxylic acid monomer as a monomer A, 58 to 99.9 mass % of an alkyl(meth)acrylate ester having an alkyl group of 4 to 12 carbon atoms as a monomer B, 30 mass % or less of an alkyl(meth)acrylate ester having 1 to 3 carbon atoms as a monomer C, and 30 mass % or less of a monomer having an ethylenically unsaturated group other than those of the monomers A to C (the total of monomers A to D is taken as 100 mass %) as a monomer D.

3. The method for producing an adhesive composition for medical use according to claim 1, wherein the gel fraction of the acrylic copolymer is 60% or higher.

4. The method for producing an adhesive composition for medical use according to claim 1, wherein the medicament having a carboxyl group is an anti-inflammatory analgesic.

5. The method for producing an adhesive composition for medical use according to claim 1, wherein the sulfuric acid content is 300 ppm or less.

6. The method for producing an adhesive composition for medical use according to claim 1, wherein the sulfuric acid content is 50 to 300 ppm.

7. The method for producing an adhesive composition for medical use according to claim 2, wherein monomer D is a vinyl monomer having a cyano group, an aromatic vinyl monomer, or a vinyl ester monomer.

8. The method for producing an adhesive composition for medical use according to claim 2, wherein the content of monomer D is 4 to 30 mass %.

9. The method for producing an adhesive composition for medical use according to claim 2, wherein the content of monomer A is 0.5 to 1.8 mass %.

10. A method for producing a patch for medical use, the method comprising:
a step of preparing an adhesive composition produced by the method for producing an adhesive composition for medical use according to claim 1, and
a step of applying the adhesive composition on one side of a substrate.
